SEQUENCE LISTING (27 sequences)

SEQ ID NO: 1 - protein, 5 aa, Artificial Sequence
  CDRH1
GYYFH

SEQ ID NO: 2 - protein, 17 aa, Artificial Sequence
  CDRH2
RIDPEDDSTKYAERFKD

SEQ ID NO: 3 - protein, 17 aa, Artificial Sequence
  CDRH3
WRIYRDSSGRPFYVMDA

SEQ ID NO: 4 - protein, 11 aa, Artificial Sequence
  CDRL1
LASEDIYTYLT

SEQ ID NO: 5 - protein, 7 aa, Artificial Sequence
  CDRL2
GANKLQD

SEQ ID NO: 6 - protein, 9 aa, Artificial Sequence
  CDRL3
LQGSKFPLT

SEQ ID NO: 7 - protein, 456 aa, Artificial Sequence
  H1
QVQLVQSGAEVKKPGASVKVSCKVSGEISTGYYFHWVRQAPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 456 aa, Artificial Sequence
  H2
QVQLVQSGAEVKKPGASVKVSCKVSGEISTGYYFHWVRRRPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 456 aa, Artificial Sequence
  H3
QVQLVQSGAEVKKPGASVKVSCKVSGEISTGYYFHFVRRRPGKGLEWMGRIDPEDDSTKYAERFKDRVTMTADTSTDTAYMELSSLRSEDTATYFCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  L1
DIQMTQSPSSVSASVGDRVTITCLASEDIYTYLTWYQQKPGKAPKLLIYGANKLQDGVPSRFSGSGSGTDFTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, Artificial Sequence
  L2
DIQMTQSPSSVSASVGDRVTITCLASEDIYTYLTWYQQKPGKAPKLLIYGANKLQDGVPSRFSGSGSGTDYTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, Artificial Sequence
  L3
DIQMTQSPSSVSASVGDRVTITCLASEDIYTYLTWYQQKPGKAPQLLIYGANKLQDGVPSRFSGSGSGTDYTLTISSLQPEDEGDYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - DNA, 23 bp, Artificial Sequence
  TaqMan forward primer for GADPH
caaggtcatccatgacaactttg

SEQ ID NO: 15 - DNA, 20 bp, Artificial Sequence
  TaqMan reverse primer for GADPH
gggccatccacagtcttctg

SEQ ID NO: 16 - DNA, 26 bp, Artificial Sequence
  TaqMan probe sequence for GADPH
accacagtccatgccatcactgccat

SEQ ID NO: 17 - DNA, 21 bp, Artificial Sequence
  TaqMan forward primer for Bactin
gagctacgagctgcctgactg

SEQ ID NO: 18 - DNA, 25 bp, Artificial Sequence
  TaqMan reverse primer for Bactin
gtagtttcgtggatgccacaggact

SEQ ID NO: 19 - DNA, 27 bp, Artificial Sequence
  TaqMan probe sequence for Bactin
catcaccattggcaatgagcggtttcc

SEQ ID NO: 20 - DNA, 21 bp, Artificial Sequence
  TaqMan forward primer for Cyclophilin
catctgcactgccaagactga

SEQ ID NO: 21 - DNA, 24 bp, Artificial Sequence
  TaqMan reverse primer for Cyclophilin
ccacaatattcatgccttctttca

SEQ ID NO: 22 - DNA, 25 bp, Artificial Sequence
  TaqMan probe sequence for Cyclophilin
ccaaacaccacatgcttgccatcca

SEQ ID NO: 23 - DNA, 20 bp, Artificial Sequence
  TaqMan forward primer for TNF-alpha
ggtgcttgttcctcagcctc

SEQ ID NO: 24 - DNA, 19 bp, Artificial Sequence
  TaqMan reverse primer for TNF-alpha
caggcagaagagcgtggtg

SEQ ID NO: 25 - DNA, 23 bp, Artificial Sequence
  TaqMan probe sequence for TNF-alpha
ctccttcctgatcgtggcaggcg

SEQ ID NO: 26 - DNA, 19 bp, Artificial Sequence
  SybrMan forward primer sequence for IL17A
cgcaatgaggaccctgaga

SEQ ID NO: 27 - DNA, 19 bp, Artificial Sequence
  SybrMan reverse primer sequence for IL17A
acgttcccatcagcgttga

(19) United States
(12) Patent Application Publication
(10) Pub. No.: US 2011/0150871 A1
(43) Pub. Date: Jun. 23, 2011

(54) TREATMENT OF AN AUTOIMMUNE DISEASE USING IL-18 ANTAGONISTS

(75) Inventors: Philip Overend, Essex (GB); Juliet Louise Reid, Hertfordshire (GB); Maria De Los Angeles Rocha Del Cura, Hertfordshire (GB); Adam Rene Taylor, Hertfordshire (GB)

(73) Assignee: GLAXO GROUP LIMITED, Greenford, Middlesex (GB)

(21) Appl. No.: 13/059,435

(22) PCT Filed: Aug. 14, 2009
(86) PCT No.: PCT/EP2009/060573

§ 371 Date: Feb. 17, 2011

Related U.S. Application Data
(60) Provisional application No. 61/089,623, filed on Aug. 18, 2008.

(51) Int. Cl. A61K 39/395; C07K 16/24; A61P 37/00

(52) U.S. Cl. 424/133.1; 424/145.1; 530/387.3

(57) ABSTRACT

The present invention relates to the field of autoimmune diseases, including rheumatoid arthritis (RA) and inflammatory bowel disease (IBD). Specifically, the invention relates to methods of treating autoimmune diseases in patients that are non-responsive or refractory over time to treatment with TNF-α antagonists and/or T-cell co-stimulation antagonists with an IL-18 antagonist.

### FIELD OF THE INVENTION

The present invention relates to the field of autoimmune diseases, including rheumatoid arthritis (RA) and inflammatory bowel disease (IBD). Specifically, the invention relates to methods of treating autoimmune diseases in patients that are non-responsive or refractory over time to treatment with TNF-α antagonists and/or T-cell co-stimulation antagonists with an IL-18 antagonist.

### BACKGROUND OF THE INVENTION

Autoimmune diseases are a group of diseases characterised by an abnormal immune system attack on an individual's own body tissue. In autoimmune diseases, normal “self” molecules of the body are mistakenly recognized by the body's own antibodies and are targeted for destruction. In addition, the body's defence system inappropriately triggers an inflammatory response, despite the lack of “foreign” substances to attack.

Rheumatoid arthritis (RA) is a chronic, systemic, inflammatory autoimmune disease that leads to progressive joint damage. RA affects more than 1% of the United States population and, in general, affects 0.5-1% of the population in the industrialised world. It is globally the most common chronic inflammatory polyarthritis (Pollard et al., 2005). Virtually all peripheral joints can be affected by the disease; however, the most commonly involved joints are those of the hands, feet and knees. RA is characterized by redness, warmth, swelling and pain. Those afflicted with RA frequently endure stiffness, especially in the morning or after long periods of sitting, and fatigue. As the disease progresses joint deformation and disability are common.

The clinical success of tumour necrosis factor-α (TNF-α) blocking biologics in a growing number of immune-mediated pathologies, including RA, Crohn's disease and psoriasis, confirms the importance of TNF-α driving chronic inflammation and represents an important step forward in alleviating the suffering caused by these conditions.

TNF-α is a key cytokine involved in inflammatory and immune responses. A number of biological agents targeting TNF-α have been licensed for clinical use (Maini and Taylor, 2000). These include the monoclonal antibodies (mAbs) infliximab (Remicade™—a chimeric mAb comprising a human IgG1κ antibody with a mouse Fv), and adalimumab (Humira™—a “human” antibody produced by phage display), and the soluble TNF-α receptor fusion protein etanercept (Enbrel™—an engineered p75 TNF-α receptor dimer linked to the Fc portion of human IgG1). These agents act as competitive inhibitors of TNF-α to its receptors. Other anti-TNF-α agents with proven clinical efficacy include CDP571, a humanised murine complementarity determining region-3 engrafted anti-TNF-α mAb; Celltech/Pharmacia's PEGylated CDP870 anti-TNF-α antibody (Choy et al., 2002); and Amgen's PEGylated anti-TNF-α receptor antibody (Davis et al., 2000).

There is a need for additional and alternative therapies for RA and other autoimmune diseases. Approximately 20% of patients given TNF-α blocking agents fail to respond (Kremer, 2001) and a significant number of additional patients become refractory to anti-TNF-α therapy over time (Durez et al., 2005), enforcing the need for further therapeutic strategies. Data exists to suggest that up to 50% of patients are no longer taking anti-TNF-α therapies after 5 years. At present it is unclear why patients do not respond to anti-TNF-α therapy, or why patients who respond initially then lose that response.

Infliximab, which is infused intravenously, has underperformed relative to adalimumab and etanercept, which are both administered by single injection (Moreland et al., 2006). Furthermore, anti-TNF-α treatment is associated with an increased susceptibility to infection. In particular, reactivation of latent tuberculosis has been a problem which has resulted in patients having to be screened before the commencement of treatment (Keane et al, 2001). Etanercept and infliximab have also been associated with nervous system disorders, such as demyelination leading to multiple sclerosis (MS), whilst infliximab is also linked to higher incidences of congestive heart failure.

As a consequence of the problems associated with anti-TNF-α therapy, investigators have sought to target other pathogenic elements of RA, and other autoimmune and inflammatory disorders, using novel biological therapies.

Abatacept (Orencia™) was the first immunotherapy directed against the process of T-cell co-stimulation. It has been approved by the US Food and Drug Administration (FDA) for the treatment of patients with RA who have had an inadequate response to other drugs, such as methotrexate or TNF-α antagonists. Abatacept has shown clinical effectiveness in RA by improving disease activity, quality of life measures and radiographic progression of disease.

Abatacept is a fully human, soluble fusion protein that consists of the extracellular domain of the human cytotoxic T-lymphocyte-associated antigen-4 (CTLA-4) linked to the modified Fc portion of human IgG1. By binding to CD80 and CD86 (B7 molecules) on the antigen-presenting cell (APC) and preventing interaction with CD28 on the T-cell, abatacept inhibits one of the key co-stimulatory pathways required for full T-cell activation. It is widely accepted that activated T-cells play a key role in orchestrating immune processes that lead to the chronic inflammation seen in the rheumatoid synovium of RA patients (Choy and Panayi, 2001). This is achieved through cell-to-cell contact and the involvement of a number of different cytokines from monocytes, macrophages and synovial fibroblasts. The release of these cytokines, which include TNF-α, IL-1 and IL-6, seems to be the pivotal event leading to the chronic inflammation characteristic of RA. Therefore, by modulating events upstream of T-cell activation, abatacept acts earlier in the immune cascade than traditional therapies and TNF-α antagonists, and has the potential to affect multiple downstream pathways and events in the immunopathology of RA (Weisman et al., 2004).

Like infliximab, abatacept is administered by intravenous infusion, which reduces the convenience of administration and may increase infection risk. The most frequently reported adverse events with abatacept are headache, upper respiratory tract infection, nasopharyngitis and nausea. Serious infections have been reported in 3% of patients (Lundquist, 2007).

IL-18, formerly called interferon-γ (IFN-γ ) inducing factor (IGIF), is a proinflammatory cytokine that plays an important role in the T-helper cell, type 1 (Th1) response. IL-18 exhibits biological activities that include the induction of TNF-α production by T-cells and NK-cells. The role of IL-18 in the development of autoimmune and inflammatory diseases has been demonstrated. IL-18 expression is significantly increased in the pancreas and spleen of the non-obese diabetic (NOD) mouse immediately prior to the onset of disease (Rothe et al., 1997). Furthermore, it has been demonstrated that IL-18 administration increases the clinical severity of murine experimental allergic encephalomyelitis (EAE), a Th1-mediated autoimmune disease that is a model for MS. In addition, it has been shown that neutralizing anti-rat IL-18 antiserum prevents the development of EAE in female Lewis rats (Wildbaum et al., 1996). Taniguchi et al. (1997) describe seven murine and six rat anti-human IL-18 monoclonal antibodies (mAbs), which bind to four distinct antigenic sites. One of the murine mAbs (#125-2H), and the six rat mAbs inhibit IL-18-induced IFN-γ production by KG-1 cells, with the rat mAbs exhibiting neutralizing activities 10-fold lower than that of #125-2H.

European patent EP0712931 discloses two mouse anti-human IL-18 mAbs, H1 (IgG1) and H2 (IgM). As demonstrated by Western blot analysis, both mAbs react with membrane-bound human IL-18, but not with membrane-bound human IL-12. H1 is utilized in an immunoaffinity chromatography protocol to purify human IL-18, and in an ELISA to measure human IL-18. H2 is utilized in a radioimmunoassay to measure human IL-18.

WO01/58956 and WO2005/047307 disclose antibodies that bind human IL-18, as well as methods of making and using such antibodies for RA.

WO2007/137984, which is expressly incorporated herein by reference in its entirety, discloses humanised anti-IL-18 antibodies, methods of manufacture and methods of treatment with said antibodies. Further disclosed are screening methods using, for example, surface plasmon resonance to identify antibodies with therapeutic potential.

### SUMMARY OF THE INVENTION

The present invention provides, in a first aspect, a method of treating an autoimmune disease in a subject that is non-responsive or refractory to treatment with a TNF-α antagonist and/or a T-cell co-stimulation antagonist, said method comprising the step of administering to the subject a therapeutically effective amount of an IL-18 antagonist.

In an embodiment of the invention the autoimmune disease is inflammatory bowel disease (IBD), psoriasis, type I diabetes, MS, or an arthritic disease such as rheumatoid arthritis (RA). In a specific embodiment, the autoimmune disease is RA.

In an embodiment of the invention the TNF-α antagonist is infliximab (Remicade™), etanercept (Enbrel™), adalimumab (Humira™), CDP571, CDP870, or CNTO148 (golimumab). In another embodiment the TNF-α antagonist is adalimumab (Humira™).

In an embodiment of the invention the T-cell co-stimulation antagonist is abatacept (Orencia™).

In a further embodiment of the invention, the IL-18 antagonist is an antibody immunospecific for IL-18.

Particular anti-IL-18 antibodies of the invention are those described in U.S. Pat. No. 6,706,487 and WO2007/137984, the contents of which are incorporated by reference herein in their entirety. Thus, in an embodiment of the present invention the anti-IL-18 antibody is a humanised antibody comprising a heavy chain and light chain having the following complementarity determining regions (CDRs): CDRH1: SEQ ID NO:1; CDRH2: SEQ ID NO:2; CDRH3: SEQ ID NO:3; CDRL1: SEQ ID NO:4; CDRL2: SEQ ID NO:5; CDRL3: SEQ ID NO:6. In a further embodiment of the invention one or more of the CDRs may be replaced by a variant thereof

CDRs and framework regions (FR) and numbering of amino acids follow, unless otherwise indicated, the Kabat definition as set forth in Kabat et al “Sequences of Proteins of Immunological Interest”, 4th Ed., U.S. Department of Health and Human Services, National Institutes of Health (1987).

The anti-IL-18 antibody of the invention may comprise a heavy chain selected from the group consisting of: SEQ ID NO:7 (H1), SEQ ID NO:8 (H2), SEQ ID NO:9 (H3); and a light chain selected from the group consisting of: SEQ ID NO:10 (L1), SEQ ID NO:11 (L2), SEQ ID NO:12 (L3). All functional combinations of the aforementioned heavy chain and light chain sequences, which bind to IL-18, are envisaged. In particular, the anti-IL-18 antibody of the invention may comprise one of:
a humanised antibody having a heavy chain of SEQ ID NO:7 (H1) and a light chain of SEQ ID NO:11 (L2) or a heavy chain of SEQ ID NO:7 (H1) and a light chain of SEQ ID NO:12 (L3);
a humanised antibody comprising a heavy chain of SEQ ID NO:8 (H2) and a light chain of SEQ ID NO:11(L2) or a heavy chain of SEQ ID NO:8 (H2) and a light chain of SEQ ID NO:12(L3).
a humanised antibody comprising a heavy chain of SEQ ID NO:9 (H3) and a light chain of SEQ ID NO:11 (L2) or a heavy chain of SEQ ID NO:9 (H3) and a light chain of SEQ ID NO:12 (L3).

In particular, the anti-IL-18 antibody may comprise a heavy chain of SEQ ID NO:7 (H1) and a light chain of SEQ ID NO:11 (L2). This antibody is referred to herein as H1L2.

It is well recognised in the art that amino acids are divided into groups based on common side-chain properties, as shown below, and that certain amino acid substitutions within these groups are regarded as being “conservative”, e.g., substituting one hydrophobic amino acid for an alternative hydrophobic amino acid, e.g., substituting leucine with valine, or isoleucine. Accordingly, sequences identified herein that further have one or more conservative amino acid substitutions are considered within the scope of the invention.

| Side chain | Members |
|---|---|
| hydrophobic | met, ala, val, leu, ile |
| neutral hydrophilic | cys, ser, thr |
| acidic | asp, glu |
| basic | asn, gln, his, lys, arg |
| residues that influence chain orientation | gly, pro |
| aromatic | trp, tyr, phe |

In an embodiment of the present invention, the anti-IL-18 antibody is an antibody that competes with an antibody comprising a heavy chain having the sequence set forth in SEQ ID NO:7 (H1) and a light chain having the sequence set forth in SEQ ID NO:11 (L2) for binding to human IL-18 in an ELISA assay.

The person skilled in the art appreciates that in order for an antibody (antibody A) to compete with an antibody comprising a heavy chain having the sequence set forth in SEQ ID NO:7 and a light chain having the sequence set forth in SEQ ID NO:11 (antibody B) for a specific binding site (human IL-18), antibody A must be present in a sufficient amount to have an effect in said assay. For example, antibody A and antibody B may be present in equimolar amounts. If antibody A is a competing antibody, the presence of antibody A may reduce the binding of antibody B to human IL-18 in an ELISA assay by more than 10%, 20%, 30%, 40% or 50%. A competing antibody (antibody A) reduces the binding of antibody B to plate bound human IL-18, whereas a non-IL-18-specific control does not. In such ELISA assays human IL-18 may be bound to an immunoassay plate.

In another embodiment of the invention the anti-IL-18 antibody comprises heavy and/or light chains comprising polypeptides which are at least 90%, 95%, 96%, 97%, 98% or 99% identical to the amino acid sequences of SEQ ID NO:7 (H1) and SEQ ID NO:11 (L2), respectively, wherein said antibody binds human IL-18.

In another embodiment of the invention IL-17A expression is down-regulated in a subject treated with an IL-18 antagonist. The down-regulation of IL-17A may be determined, for example, using quantitative PCR or transcriptomics.

In an aspect of the invention, there is provided an IL-18 antagonist for use in the treatment of an autoimmune disease in a subject that is non-responsive or refractory to treatment with a TNF-α antagonist and/or a T-cell co-stimulation antagonist.

In another aspect of the invention, there is provided an IL-18 antagonist for use in down-regulating IL-17A expression or activity in a subject afflicted with an autoimmune disease.

Also included in the invention are polynucleotide sequences encoding the aforementioned antibodies.

### BRIEF DESCRIPTION OF THE DRAWINGS

FIG. 1 is a schematic outlining the experimental design of Example 1. 6 donors were recruited for this study. 4-5 biopsies were taken from each patient. Each biopsy was then divided into 4 explants and cultured for 3 days ex-vivo in the presence of either: Synagis™ control IgG (4 μg/ml), Humira™ (4 μg/ml), H1L2 (4 μg/ml) or Abatacept (4 μg/ml). Note: Explants from 2 patients did not receive Abatacept treatment, therefore the final sample numbers for each treatment group were as follows: control IgG (26), Humira™(26), H1L2 (26), Abatacept (18).

FIG. 2 is a Venn diagram summarising the microarray gene significant differential (P≦0.05) expression changes, observed in the synovial explant studies of Example 1, for H1L2, Humira™ and abatacept treatment versus Synagis™ control.

FIG. 3 shows the mean ratio of gene expression in treated synovial explant samples to gene expression in control synovial explant samples, with a 95% confidence interval, for selected transcripts that are regulated by H1L2 treatment: A) TIMP4, B) Noggin, C) EGF, D) TNF-α, E) IL-18RAP, F) ITGA6. Where the confidence intervals do not cross the 1.0 line, this indicates that the treatment significantly (P≦0.05) changed the expression of the gene compared to the control.

FIG. 4 shows the mean ratio of gene expression in treated synovial explant samples to gene expression in control synovial explant samples, with a 95% confidence interval calculated from the unadjusted (raw) p value, for IL17A. Where the confidence interval does not cross the 1.0 line, this indicates that the treatment significantly (P≦0.05) changed the expression of IL17A compared to the control. The table shows the fold change, raw and Dunnett's adjusted p values for comparison.

FIG. 5 shows least squares mean (LS-mean) plots in respect of the modelled gene expression intensity for each treatment group (Synagis™ control, H1L2, Humira™, and abatacept), with a 95% confidence interval, for selected transcripts that are regulated by H1L2 treatment: A) Noggin, B) EGF, C) TNF-α, D) IL-18RAP, E) TIMP4 and F) ITGA6

### DETAILED DESCRIPTION OF THE INVENTION

Surprisingly, it has now been found that an anti-IL-18 antibody modulates the expression of a different subset of genes compared with the TNF-α antagonist adalimumab and the T-cell co-stimulation antagonist abatacept. More surprisingly still, IL-17A is among the subset of genes specifically modulated by the anti-IL-18 antibody. These observations suggest, for the first time, that an IL-18 antagonist may be effective in the treatment of those patients who are non-responsive or refractory over time to treatment with a TNF-α antagonist or a T-cell co-stimulation antagonist.

An “autoimmune disease” is a disease or disorder arising from and directed against an individual's own tissues. Examples of autoimmune diseases or disorders include, but are not limited to, arthritis and other arthritic diseases (including rheumatoid arthritis (RA), juvenile rheumatoid arthritis, osteoarthritis and psoriatic arthritis), type 1 diabetes, psoriasis, inflammatory bowel disease (IBD) including Crohn's disease and ulcerative colitis (UC), systemic lupus erythematosus (SLE, Lupus), Sjögren's syndrome, scleroderma, vasculitides (including Takayasu arteritis, giant cell (temporal) arteritis, polyarteritis nodosa, Wegener's granulomatosis, Kawasaki disease, isolated CNS vasculitis, Churg-Strauss arteritis, microscopic polyarteritis/polyangiitis, hypersensitivity vasculitis (allergic vasculitis), Henoch-Schonlein purpura, and essential cryoglobulinemic vasculitis), undifferentiated spondyloarthropathy (USpA), ankylosing spondylitis (AS), graft-versus-host disease (GVHD), primary biliary cirrhosis (PBC), primary sclerosing cholangitis (PSC), idiopathic thrombocytopenic purpura (ITP), idiopathic pulmonary fibrosis (IPF), multiple sclerosis (MS), and asthma. Any one or more of the aforementioned autoimmune diseases may be the target disease for a method of treatment of the invention. In a particular embodiment, the autoimmune disease is selected from the group consisting of IBD, psoriasis, type I diabetes, MS, and an arthritic disease such as RA. Particularly, the autoimmune disease is RA or IBD. In another embodiment, the autoimmune disease is RA.

A “TNF-α antagonist” is an agent that inhibits or antagonises, to some extent, a biological activity of TNF-α. This antagonism may be achieved by preventing TNF-α from binding to its receptors or reducing said binding. Agents that bind to TNF-α and neutralise its activity, such as TNF-α binding proteins, are included, as are agents that bind to and neutralise the activity of soluble or membrane bound TNF-α receptors. TNF-α antagonists which are specifically contemplated include infliximab (Remicade™), etanercept (Enbrel™), adalimumab (Humira™), CDP571, CDP870 and CNTO148 (golimumab). A “TNF-α antagonist” as defined herein is not intended to encompass an “IL-18 antagonist”. For the avoidance of doubt, all “IL-18 antagonists” as defined below are specifically excluded from the definition of “TNF-α antagonist” as used herein.

A “T-cell co-stimulation antagonist” is an agent that is capable of reducing or preventing T-cell activation by reducing or preventing the T cell co-stimulatory signal. T cells require at least two signals for full activation: an antigen-specific signal (signal 1) and a co-stimulatory signal (signal 2). A T-cell activation co-stimulation antagonist, therefore, interferes with the second signal. The first signal involves the interaction between a T-cell receptor on the T-cell with a major histocompatability complex (MHC) molecule on the antigen presenting cell (APC). The second, or co-stimulatory signal, involves the interaction between CD28 on the T-cell and CD80/86 (B7 molecules) on the APC. An example of T-cell co-stimulation antagonist is abatacept (Orencia™).

An “IL-18 antagonist” is an agent that inhibits or antagonises, to some extent, a biological activity of IL-18. IL-18 antagonists include agents which bind to IL-18, such as IL-18 binding proteins (IL-18BP) including IL-18BP-Fc fusion proteins, or antagonists which bind to a receptor for IL-18 and thereby prevent IL-18 from exerting its biological activity. Specifically contemplated IL-18 antagonists are anti-IL-18 antibodies that are immunospecific for IL-18, and that antagonise an activity of IL-18. Non-limiting examples of IL-18 antagonists include H1 and H2 described in European patent EP0712931, H18-108 (Hamasaki et al., 2005), and the antibodies described in WO01/58956, WO2005/047307 and WO2007/137984.

The term “anti-IL-18”, as it refers to antibodies of the invention, means that such antibodies are capable of neutralising a biological activity of human IL-18. It does not exclude, however, that such antibodies may also in addition neutralise the biological activity of non-human primate (e.g. rhesus and/or cynomolgus) IL-18 and/or forms of IL-18 present in other species.

The term “antibody” is used herein in its broadest sense and includes monoclonal antibodies (including full length monoclonal antibodies), polyclonal antibodies, multispecific antibodies (e.g. bispecific antibodies), and antibody fragments thereof (including Fab, Fd, Fab′, F(ab′)₂, Fv, ScFv fragments, and immunoglobulin single variable domains (domain antibodies, dAbs)), as well as domains based on non-Ig scaffolds, so long as they retain a desired biological activity. Said antibodies may be chimeric, humanised or human.

The phrase “single variable domain” refers to an antigen binding protein variable domain (for example, VH, VHH, VL) that specifically binds an antigen or epitope independently of a different variable region or domain.

A “domain antibody” or “dAb” may be considered the same as a “single variable domain” which is capable of binding to an antigen. A single variable domain may be a human antibody variable domain, but also includes single antibody variable domains from other species such as rodent (for example, as disclosed in WO 00/29004), nurse shark and Camelid VHH dAbs. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “domain antibodies”. As used herein VH includes camelid VHH domains.

A “domain based on a non-Ig scaffold” includes a domain which is a derivative of a scaffold selected from the group consisting of CTLA-4, lipocalin, SpA, an Affibody, an avimer, GroE1, transferrin, GroES and fibronectin/adnectin, which has been subjected to protein engineering in order to obtain binding to an antigenother than the natural ligand.

The term “immunospecific” as used in relation to an antibody means an antibody that binds its target protein (e.g. human IL-18) with no or insignificant binding to other proteins. The term, however, does not exclude the fact that an antibody to a target protein in a given species (e.g. human) may also be cross-reactive with other forms of the target protein in other species (e.g. a non-human primate).

“Non-responsive to treatment with a TNF-α antagonist or a T-cell co-stimulation antagonist” refers to a subject that does not respond, or responds negatively, to treatment with a TNF-α antagonist or a T-cell co-stimulation antagonist. Accordingly, a non-responsive subject would either have a) the same disease symptoms before and after treatment with a TNF-α antagonist and/or a T-cell co-stimulation antagonist, or b) worse disease symptoms following treatment with a TNF-α antagonist and/or a T-cell co-stimulation antagonist.

“Refractory to treatment with a TNF-α antagonist or a T-cell co-stimulation antagonist” refers to an inadequate or unsustained response to a previous or current treatment with a TNF-α antagonist or a T-cell co-stimulating antagonist, respectively. A subject that is refractory to treatment with a TNF-α antagonist or a T-cell co-stimulation antagonist includes, therefore, a subject that previously responded to such treatment, but no longer responds to said treatment to the same degree. A refractory subject includes a subject whose illness relapses back to its former state, with the return of disease symptoms, following an apparent recovery or partial recovery.

Patients with an inadequate response to TNF-α antagonists typically have severe and/or longer standing disease, and are often difficult to treat, some having also had an inadequate response to non-biologic agents prior to TNF-α antagonist treatment (Lundquist, 2007).

An “inadequate or unsustained response” may be due to toxicity associated with the treatment and/or inadequate efficacy of the treatment. An inadequate response to a specific treatment may be established by studying one or more clinical markers, which are associated with the disease or disorder, known to those skilled in the art. Accordingly, an inadequate response can be determined by a clinician skilled in treating the autoimmune disease in question.

In the case where the autoimmune disease is RA, a recognised way of assessing patient response to treatment is by means of “ACR response values” and the “Disease Activity Score 28” (DAS28). The ACR response criteria objectively record clinical response to treatment and reductions in disease activity, whereas the DAS28 assesses overall disease activity (Lundquist, 2007).

RA is difficult to study for a variety of reasons. Firstly, there is a wide variability between patients: the disease can start at different ages; different joints can be afflicted by the disease; the degree and time course of destruction varies; patients can, but might not, have extra-articular manifestations early on; and up to 40% of RA patients, and even more in early disease, can be negative for rheumatoid factor (RF), the classical autoantibody in the disease, whose presence is associated with severity and destruction. Secondly, there are dozens of potential outcome measures, such as tender or swollen joint counts (TJC, SJC), morning stiffness of finger joints, pain or global disease assessments by patients or physicians etc. Thirdly, patients with RA are amenable to a tremendous placebo response of most of these variables (Smolen et al., 2003).

The American College of Rheumatology (ACR) have determined and validated core sets of clinical variables that were sensitive to change, had long-term predictive value, had little, if any, redundancy, and so were reliable in determining disease activity and/or outcome in clinical trials.

The ACR improvement criteria are as follows:
20% improvement in tender-joint count (TJC, using the 68 or 28 joint count), and
20% improvement in swollen-joint count (SJC, using the 66 or 28 joint count), and
20% improvement in three of the following: patient global assessment of disease activity (Visual Analogue Scale (VAS) or five-point Likert scale); assessors global assessment of disease activity (VAS or five-point Likert scale); patient assessment of pain (VAS); patient assessment of physical function (by health assessment questionnaire); acute phase response (erythrocyte sedimentation rate (ESR) or C-reactive protein (CRP)).

The above criteria are termed “the ACR20 response”. ACR50 and ACR70 response are as defined above, but with 50% or 70% improvement in the variables, respectively (Smolen et al., 2003).

An “inadequate response” for RA patients to treatment with a TNF-α antagonist or a T-cell co-stimulation antagonist may be defined as an ACR response of less than ACR20.

The Disease Activity Score (DAS) is a combined index to measure the disease activity in patients with RA. DAS28, which uses the 28 joint count, is defined below.

DAS28=0.56√TJC28+0.28√SJC28+0.70(In(ESR))+0.014(general health)

The DAS28 provides a number on a scale from 0 to 10 that indicates the current activity of the rheumatoid arthritis of the patient. A DAS28 of greater than 5.1 indicates high disease activity, whereas a DAS28 of less than 3.2 indicates low disease activity, with values between 3.2 and 5.1 being indicative of moderate disease activity.

Based on the DAS28, response criteria have been developed: the European League Against Rheumatism (EULAR) response criteria. The EULAR response criteria include not only change in disease activity but also current disease activity and were developed to measure individual response in clinical trials. To be classified as responders, patients should have a significant change in DAS28 and also low current disease activity. A major improvement in disease activity is defined as a reduction in DAS28 of 1.2 and a change from high to moderate or moderate to low disease activity. Minor improvement is defined as a reduction in DAS28 of 0.6 with a change between categories (high to moderate, or moderate to low). Accordingly, a change in DAS28 score of less that 0.6 is not considered to be clinically significant and is considered to be an “inadequate response” in the present context.

Despite their different constructions, the ACR improvement criteria and the EULAR response criteria were found to be in reasonable agreement in clinical trials (Fransen, 2005).

In the case where the autoimmune disease is Crohn's disease/IBD, a number of clinically validated indices exist, including Crohn's Disease Activity Index (CDAI), Perianal Crohn's Disease Activity Index (PDAI) and fistula drainage assessment, as well as quality of life scores, such as the Inflammatory Bowel Disease Questionnaire (IBDQ). There are also a number of sub-clinical markers (CRP, faecal calprotectin, intestinal permeability) and endoscopic indices (Crohn's Disease Endoscopic Index of Severity (CDEIS)/Simple Endoscopic Score for Crohn's Disease (SES-CD)) (Sostegni, 2003).

In the case where the autoimmune disease is psoriasis, lesions are quite visible and therefore relatively easy to quantify. The basic characteristics of psoriasis lesions are redness, thickness and scaliness. Unfortunately, simple quantitation of the lesions is not a complete assessment of the severity of the disease, as the impact of the lesions is experienced differently by different patients (Feldman, 2005).

A 75% reduction in the psoriasis area and severity index (PASI) score (PASI 75) is the benchmark of primary endpoints for most clinical trials of psoriasis (Carlin, 2004). The PASI is a measure of the average redness, thickness and scaliness of the lesions (each graded on a 0-4 scale), weighted by the area of involvement. Both PASI 75 and PASI 50 are considered to be clinically meaningful endpoints for clinical trials. An “inadequate response” for patients suffering from psoriasis to treatment with a TNF-α antagonist or a T-cell co-stimulation antagonist may be defined as a PASI score of less than 50 or, more stringently, a PASI score of less than 75.

Other measures, such as the Salford Psoriasis Index (SPI), the Self-Administered PASI (SA-PASI) and the National Psoriasis Foundation (NPF) Psoriasis Score (NPF-PS) have also been developed for assessing the efficacy of newly developed medications on psoriasis. Biopsies and photographs are two other ways of quantitatively measuring psoriasis (Feldman, 2005). Specific instruments that focus on aspects of quality of life that are affected by skin disease include the Dermatology Life Quality Index (DQLI) and the Skindex (Feldman, 2005).

The clinical markers and indices listed above in relation to RA, IBD and psoriasis are examples only and are in no way limiting on the invention. A clinician skilled in treating the autoimmune disease in question would be able to establish the most relevant marker or index or combination of the same for use in determining whether a response to a specific treatment was adequate or inadequate.

A “subject” refers to a vertebrate, in particular a mammal and more particularly a human. Domestic and farm animals such as dogs, horses, cats, cows, etc. are included in the definition.

The term “therapeutically effective amount” refers to an amount (dose) of a substance, e.g. an IL-18 antagonist, that is sufficient to inhibit, halt, or allow an improvement in the disease being treated, e.g. an autoimmune disease such as RA, when administered alone or in conjunction with another pharmaceutical agent or treatment in a particular subject or subject population. A “therapeutically effective amount” may be an amount giving a desired therapeutic effect e.g. eliciting a specific biological or medical response. It should be noted that a “therapeutically effective amount” of a substance need not cure a disease, but should, at least, provide treatment for a disease. A “therapeutically effective amount” can be determined experimentally in a laboratory or clinical setting, for the particular disease and subject being treated.

“Identity,” means, for polynucleotides and polypeptides, as the case may be, the comparison calculated using an algorithm provided in (1) and (2) below:
(1) Identity for polynucleotides is calculated by multiplying the total number of nucleotides in a given sequence by the integer defining the percent identity divided by 100 and then subtracting that product from said total number of nucleotides in said sequence, or:

nn≦xn−(xn·y),

wherein nn is the number of nucleotide alterations, xn is the total number of nucleotides in a given sequence, y is 0.95 for 95%, 0.97 for 97% or 1.00 for 100% etc., and · is the symbol for the multiplication operator, and wherein any non-integer product of xn and y is rounded down to the nearest integer prior to subtracting it from xn. Alterations of a polynucleotide sequence encoding a polypeptide may create nonsense, missense or frameshift mutations in this coding sequence and thereby alter the polypeptide encoded by the polynucleotide following such alterations.
(2) Identity for polypeptides is calculated by multiplying the total number of amino acids by the integer defining the percent identity divided by 100 and then subtracting that product from said total number of amino acids, or:

na≦xa−(xa·y),

wherein na is the number of amino acid alterations, xa is the total number of amino acids in the sequence, y is 0.95 for 95%, 0.97 for 97% or 1.00 for 100% etc., and · is the symbol for the multiplication operator, and wherein any non-integer product of xa and y is rounded down to the nearest integer prior to subtracting it from xa.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in full length antibody sequences and in relation to CDRs are numbered according to the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

A “CDR variant” includes a partial alteration of the CDR amino acid sequence by deletion or substitution of one to several amino acids of the CDR, or by addition or insertion of one to several amino acids to the CDR, or by a combination thereof. The CDR variant may contain 1, 2, 3, 4, 5 or 6 amino acid substitutions, additions or deletions in the amino acid sequence of the CDR. The CDR variant may contain 1, 2 or 3 amino acid substitutions, insertions or deletions in the amino acid sequence of the CDR. The substitutions in amino acid residues may be conservative substitutions, for example, substituting one hydrophobic amino acid for an alternative hydrophobic amino acid. For example leucine may be substituted with valine, or isoleucine.

Antibodies which comprise a variant CDR will have the same or similar functional properties to those comprising the CDRs discussed above. Therefore, antibodies which comprise a variant CDR will bind to the same target protein or epitope with the same or similar binding affinity to the CDR described herein.

With regard to pharmaceutical compositions of the invention, it should be appreciated that determination of proper dosage forms, dosage amounts and routes of administration is within the level of ordinary skill in the pharmaceutical and medical arts and is described below.

Purified preparations of antagonists of the invention (particularly antibodies and monoclonal preparations thereof) may be incorporated into pharmaceutical compositions for use in the treatment of human diseases and disorders such as those outlined above. Typically such compositions further comprise a pharmaceutically acceptable (i.e. inert) carrier as known and called for by acceptable pharmaceutical practice, see e.g. Remingtons Pharmaceutical Sciences, 16th ed, (1980), Mack Publishing Co. Examples of such carriers include a sterilised carrier such as saline, Ringers solution or dextrose solution, buffered with suitable buffers to a pH within a range of 5 to 8. Pharmaceutical compositions for injection (e.g. by intravenous, intraperitoneal, intradermal, subcutaneous, intramuscular or intraportal) or continuous infusion are suitably free of visible particulate matter and may comprise between 0.1 ng to 1000 mg of antagonist, typically between 5 mg and 25 mg of antagonist. Methods for the preparation of such pharmaceutical compositions are well known to those skilled in the art. Pharmaceutical compositions of the invention may comprise between 0.1 ng to 1000 mg of therapeutic antagonists, specifically antibodies of the invention in unit dosage form, optionally together with instructions for use. Pharmaceutical compositions of the invention may be lyophilised (freeze dried) for reconstitution prior to administration according to methods well known or apparent to those skilled in the art. Where embodiments of the invention comprise antibodies of the invention with an IgG1 isotype, a chelator of copper such as citrate (e.g. sodium citrate) or EDTA or histidine may be added to the pharmaceutical composition to reduce the degree of copper-mediated degradation of antibodies of this isotype, see EP 0612251.

Effective doses and treatment regimes for administering an antagonist of the invention are generally determined empirically and are dependent on factors such as the age, weight and health status of the patient and the disease or disorder to be treated. Such factors are within the purview of the attending physician. Guidance in selecting appropriate doses may be found in e.g. Smith et al (1977) Antibodies in human diagnosis and therapy, Raven Press, New York but will in general be between 0.1 mg and 1000 mg. The potential effective dose for treating a human patient afflicted with RA with H1L2 is in the order of 0.3-0.8 mg (4.3 μg/kg-11.4 μg/kg for a subject weighing 70 kg) infused intravenously for 2-hours at steady state after repeat dosing (with a dosing interval of 28 days) and based on 90% inhibition of IL-18 in the synovial fluid.

Depending on the disease or disorder to be treated, pharmaceutical compositions comprising a therapeutically effective amount of an antagonist of the invention may be used simultaneously, separately or sequentially with an effective amount of another medicament. Conveniently, a pharmaceutical composition comprising a kit of parts of an antibody of the invention together with such other medicaments optionally together with instructions for use is also contemplated by the present invention.

All references mentioned throughout this specification are expressly and entirely incorporated herein by reference.

Further details of the invention are illustrated by the following non-limiting examples.

### Example 1

### In vitro Analysis of Synovial Tissue Explants from RA Patients

### Introduction

IL-18 is present in RA synovial fluid and it is thought to contribute to pannus formation by stimulating angiogenesis and upregulating adhesion molecule expression. In addition, it stimulates IFNγ production and has regulatory effects on local production of TNF-α and IL-1β.

### Preparatory Work

Patients: RA patients with active knee involvement (i.e. RA manifested in the inflamed knee) were recruited from St. Vincent's University Hospital, Dublin.

Macroscopic Analysis: The synovial membrane (SM) was assessed macroscopically using a previously validated visual analogue scale (VAS: 0-100 mm). Vascular markings were defined by blood vessel pattern: tortuous, bushy vessels=1; and straight branching vessels=0.

Preliminary data: In preliminary experiments approximately 1 mm explants were cultured in the presence of a control murine monoclonal antibody. Sample explants were frozen at intervals and subsequently analysed by immunohistochemistry using an anti-mouse Ig antibody. The results showed that following incubation with the murine antibody at 4 μg/ml, the antibody penetrated throughout the tissue explant in 24 hours. This experiment demonstrated that the entire explant was exposed to antibody which was a pre-requisite for further studies.

### Methods for Explant Cultures

Explant biopsies were obtained from patients undergoing arthroscopic examination. Synovial tissue was visibly red, inflamed and often villous and was taken from the site of increased inflammation under direct visualisation. For each set of conditions one large SM biopsy was dissected into approximately 1 mm³ pieces, therefore ensuring that the synovial architecture and cellular infiltration were the same across a set of experimental conditions. One of the dissected biopsies from each larger biopsy was subsequently examined histologically to assess the synovial architecture.

SM explants (1 mm³) were cultured in 96 well plates in DMEM containing 10% heat inactivated fetal calf serum, penicillin and streptomycin. The SM explants were incubated in the presence or absence of neutralising H1L2, Humira™ (adalimumab, a human IgG1 anti-TNF mAb, U.S. Pat. No. 6,090,382), Synagis™ (a control human IgG1 monoclonal anti-respiratory syncitial virus (RSV) antibody) or Orencia™ (abatacept, a human IgG1 anti-CTLA-4 mAb, U.S. Pat. No. 5,851,795) at 4 μg/ml for 72 hours at 37° C. and 5% CO₂. SM explants were snap frozen and stored at −80° C. for RNA profiling analysis.

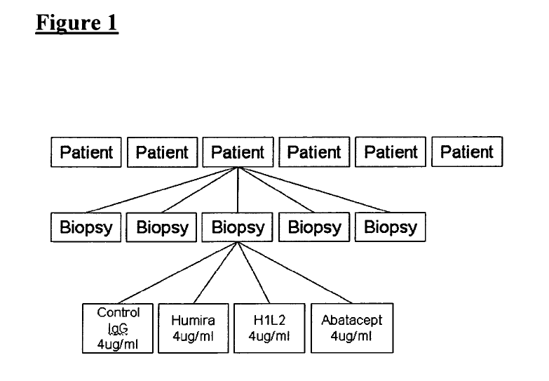

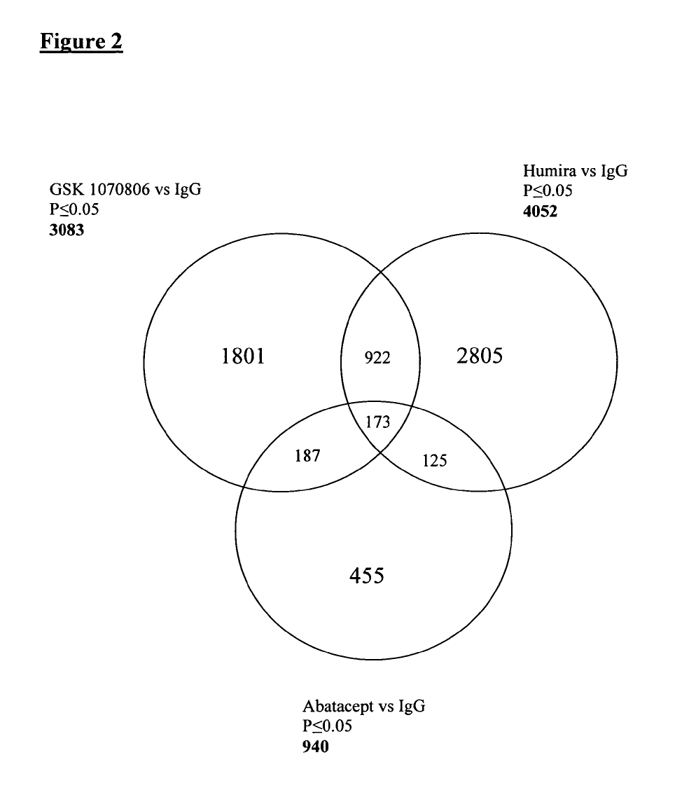

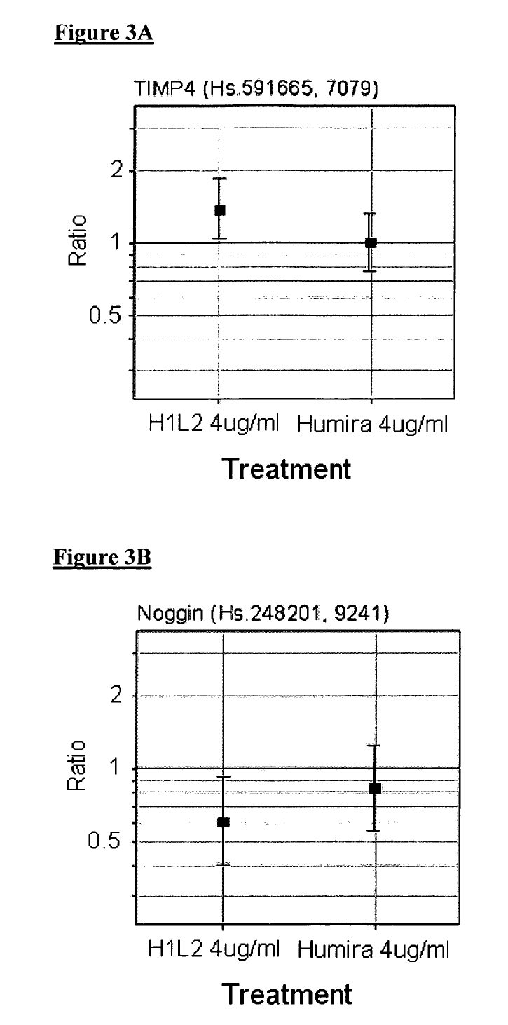

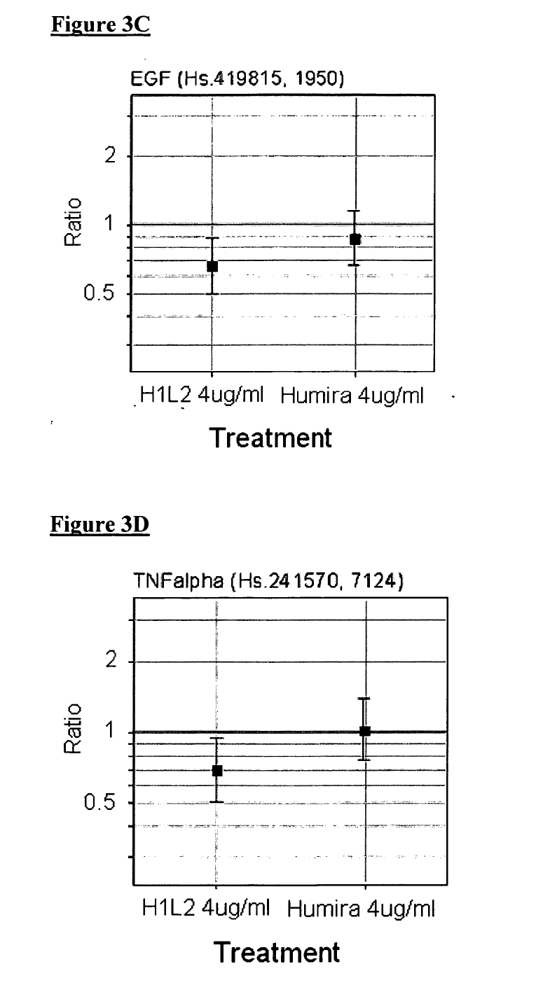

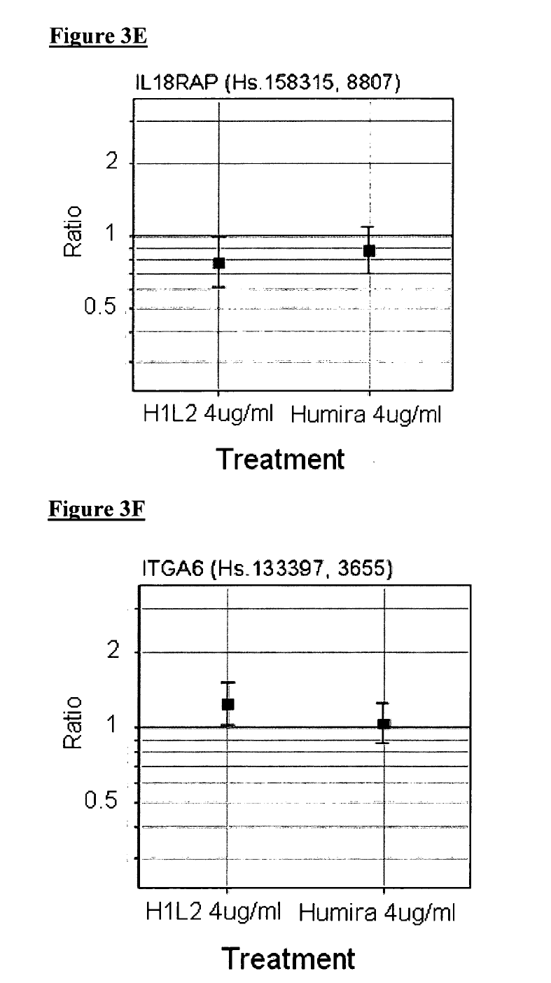

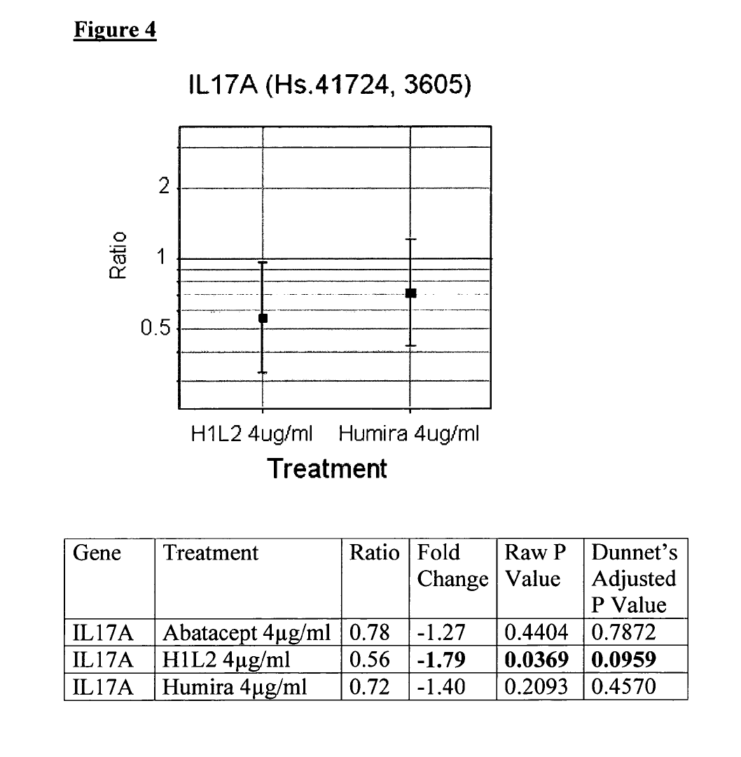

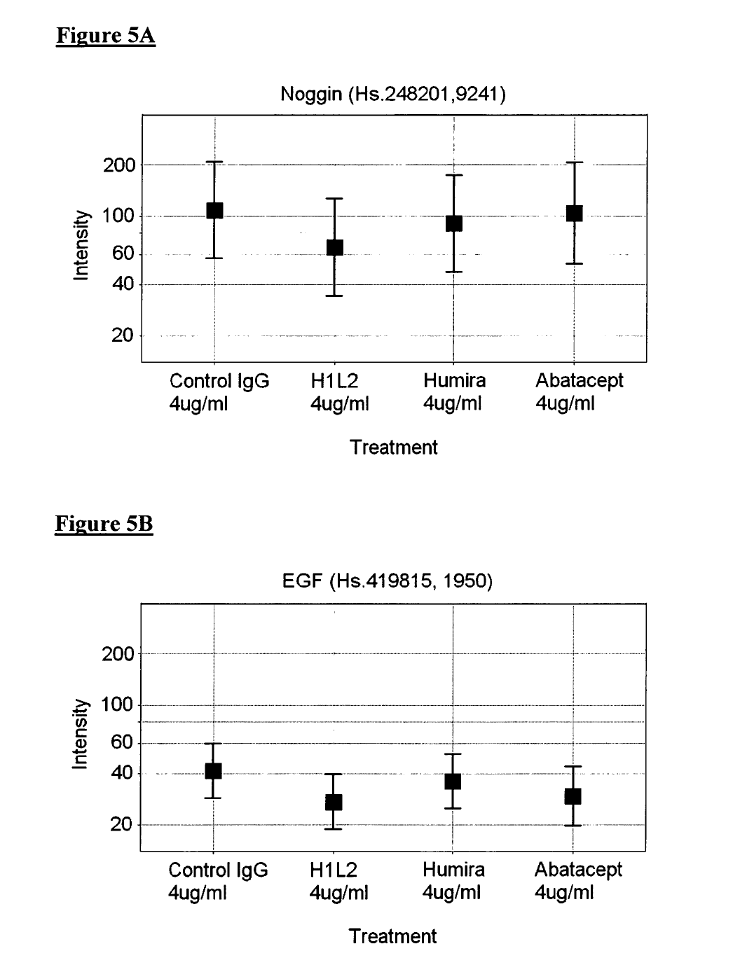

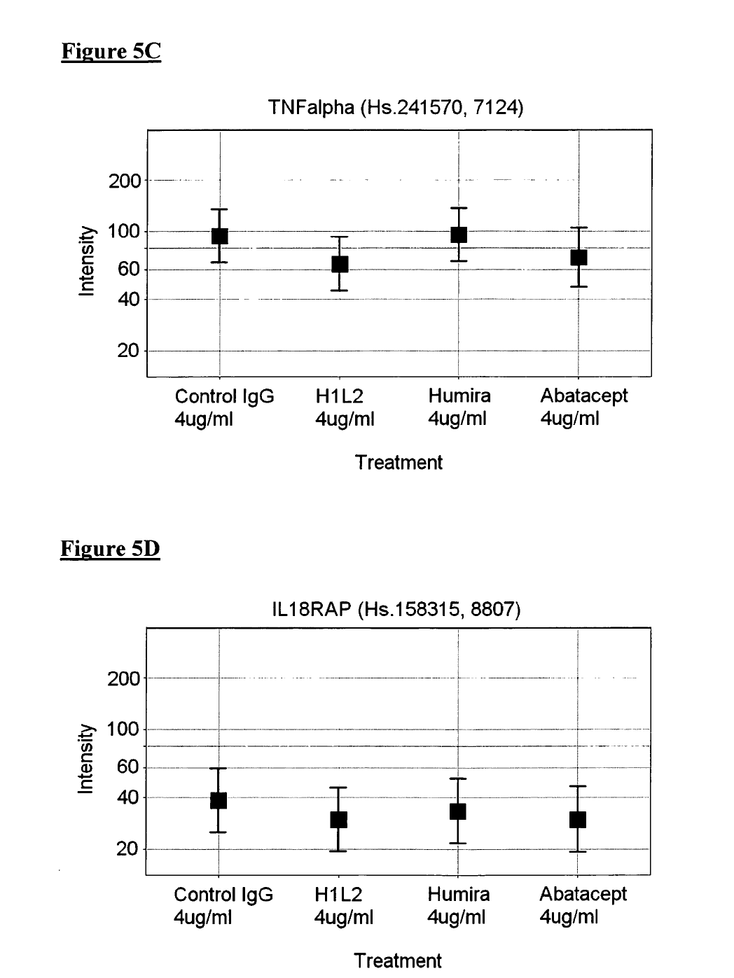

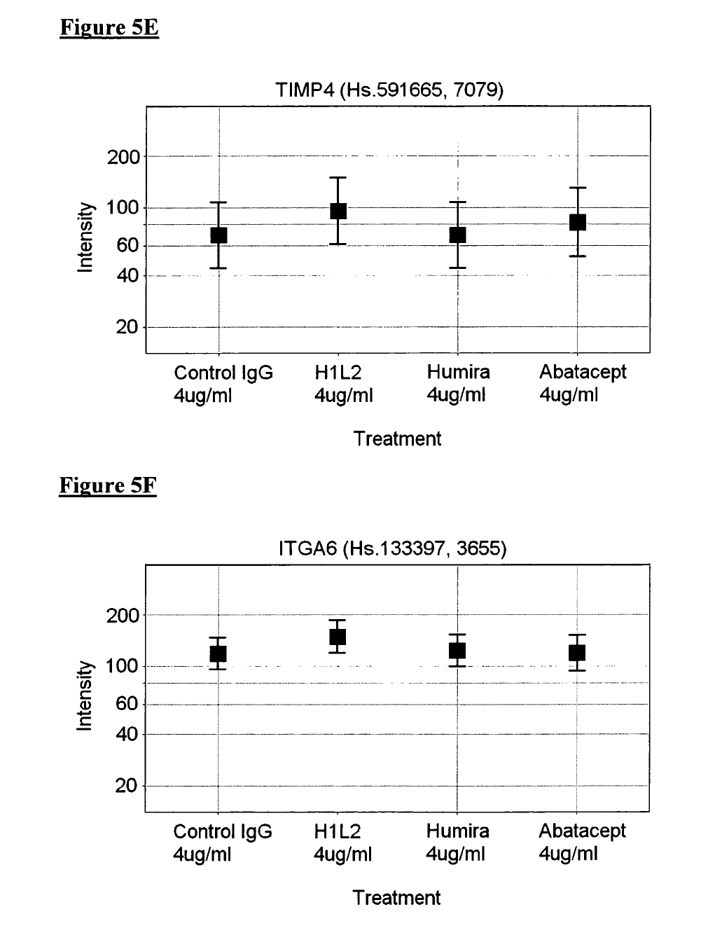

A schematic outlining the experimental design is shown in FIG. 1. Six donors were recruited for this study with 4-5 biopsies being taken from each patient. It should be noted that explants from 2 patients did not receive abatacept treatment, therefore the final sample numbers for each treatment group were as follows: control IgG (26), Humira™ (26), H1L2 (26), Abatacept (18).

### RNA Isolation

Total RNA was isolated from 96 frozen SM explants from six patients using a Polytron type homogeniser (YellowLine D1 25 Basic) and 1 ml of TriZol reagent (Invitrogen). The RNA was further purified using RNeasy mini columns (Qiagen), including on-column DNASE1 step to remove any contaminating genomic DNA, and eluted in water. The quantity of extracted RNA was determined using a NanoDrop and quality was assessed using pico chips on an Agilent 2100 bioanalyser (South Plainfield, N.J., USA). RNA yields were in the range of 166.4 to 1228.8 ng and only 3 samples failed to show visible 28S and 18S rRNA peaks on the bioanalyser. These 3 samples were excluded from further analysis.

### RNA Amplification and Labelling

The 93 samples and 3 amplification control RNAs were randomised across a 96 well plate. Samples from 3 patients were included in each half of the plate, to ensure that samples from an individual patient were kept together over the two hybridization batches. Duplicate plates of 50 ng of total RNA were converted to amplified antisense sscDNA using the NuGEN Ovation RNA Amplification System V2, including Ovation WB reagent, following manufacturer's instructions and performed on a Tecan Freedom Evo robot. cDNAs were purified using the Agencourt AMPure Magnetic Bead Purification System. The cDNA concentrations were determined by measurement on a Thermo Electron Varioskan spectral scanning multimode reader and quality was assessed by nano chips on an Agilent 2100 bioanalyser. 1 μg of cDNA from each batch was dispensed for qPCR and 3.75 μg of cDNA from batch 2 was dispensed for arrays.

### Quantitative PCR

The duplicate cDNAs were diluted to 10 ng/μl and stamped out at 2 μl/well onto 384 well plates. Plates were cycled on an Applied Biosystems 7900 machine using the TaqMan 384 default programme. All primers (Sigma-Genosys) and TaqMan probes (Biosearch Technologies) were tested under assay conditions using genomic DNA as a template in ten-fold serial dilution from 10000 down to 1 copy.

TaqMan reactions, using TaqMan Universal PCR mastermix (Applied Biosystems) according to manufacturer's instructions, were completed for GAPDH, Bactin, Cyclophilin and TNF-alpha. A SYBR green reaction, using SYBR green PCR mastermix (Applied Biosystems) according to manufacturer's instructions, was completed for IL17A. The data was collected using SDS v2.2.2 and the resulting CTs were exported to Excel for further analysis. A copy number per 20 ng of total RNA was generated for each replicate. Mean copies per 20 ng total RNA were then calculated and tabulated for further analysis. A single H1L2 treated biopsy failed to generate any reliable data and was excluded from any further analysis.

Primers and probe (where used) sequences for qPCR:

TaqMan primer and probe sequences for GAPDH
(Hs. 544577, 2597)
(SEQ ID NO: 14)
Forward primer: CAAGGTCATCCATGACAACTTTG
(SEQ ID NO: 15)
Reverse primer: GGGCCATCCACAGTCTTCTG
(SEQ ID NO: 16)
Probe: ACCACAGTCCATGCCATCACTGCCAT
TaqMan primer and probe sequences for Bactin
(Hs. 520640, 60)
(SEQ ID NO: 17)
Forward primer: GAGCTACGAGCTGCCTGACTG
(SEQ ID NO: 18)
Reverse primer: GTAGTTTCGTGGATGCCACAGGACT
(SEQ ID NO: 19)
Probe: CATCACCATTGGCAATGAGCGGTTTCC
TaqMan primer and probe sequences for Cyclophilin
(Hs. 356331, 5478)
(SEQ ID NO: 20)
Forward primer: CATCTGCACTGCCAAGACTGA
(SEQ ID NO: 21)
Reverse primer: CCACAATATTCATGCCTTCTTTCA
(SEQ ID NO: 22)
Probe: CCAAACACCACATGCTTGCCATCCA
TaqMan primer and probe sequences for TNF-α
(Hs. 241570, 7124)
(SEQ ID NO: 23)
Forward primer: GGTGCTTGTTCCTCAGCCTC
(SEQ ID NO: 24)
Reverse primer: CAGGCAGAAGAGCGTGGTG
(SEQ ID NO: 25)
Probe: CTCCTTCCTGATCGTGGCAGGCG
SybrMan primer sequences for IL17A
(Hs. 41724, 3605)
(SEQ ID NO: 26)
Forward primer: CGCAATGAGGACCCTGAGA
(SEQ ID NO: 27)
Reverse primer: ACGTTCCCATCAGCGTTGA

### Affymetrix Microarrays

3.75 μg of amplified cDNA was fragmented and biotin-labelled using the FL-Ovation cDNA Biotin Module V2 (NuGEN Technologies) and hybridization cocktails were prepared in accordance with NuGEN protocols. Cocktails were then hybridized to U133plus2.0 whole genome GeneChips in two batches, in accordance with Affymetrix protocols. GeneChips were scanned on GeneChip 3000 scanners and the fluorescence intensity for each feature of the array was obtained using the GeneChip Operating Software (Affymetrix). A total of 93 samples were hybridised and standard Affymetrix quality control criteria were assessed. One sample (H1L2 treated biopsy) gave poor quality QC metrics (Bactin 3′5′ ratio of 20256, percent present 40.30) and was excluded from further analysis. Backgrounds for the remaining samples were in the range of 27.12-43.15 and the mean percent detected probe sets across the arrays was 65.97 reflecting the high quality of the data. All probe sets were mapped to genes using Affymetrix standard annotation through NetAffx (www.affymetrix.com).

### Quantitative PCR Data Analysis

The data was log₁₀ transformed for all analysis. Housekeeper gene expression (GAPDH, Bactin and Cyclophilin) was tested for treatment effects using analysis of variance. GAPDH showed a significant effect of treatment and was therefore excluded as a normalisation factor. The first principle component from a Principle Component Analysis (Simca-P v11) of Bactin and Cyclophilin was used as a covariate for RNA loading normalisation. The data for TNF-alpha and IL17A was then analysed in SASv9.1 by mixed model analysis of variance with treatment as a main factor, the principle component of the housekeepers as a covariate and patient and biopsy (nested within patient) as random factors. Estimates for each treatment compared to baseline (Synagis™ control IgG treatment group) were calculated. Fold changes and both raw and Dunnett's adjusted P values were determined.

### Transcriptomics Data Analysis

The raw signal intensities (.Cel files) for each scan were imported into the gene expression analysis software Resolver v5.1 (Rosetta Biosoftware, Seattle, USA). Signal extraction was performed within Resolver and the normalised data was then exported for further analysis. All probesets (54,613) were log₁₀ transformed (values <1 were floored to 1) and were analysed in SASv9.1 by mixed model analysis of variance with treatment as a main factor and patient and biopsy (nested within patient) as random factors. Estimates for each treatment compared to baseline (Synagis™ control IgG treatment group) were calculated and fold changes and unadjusted P values were determined. The data was filtered to remove poorly detected probesets (defined as probesets with a median intensity <30 in all treatment groups) and poorly designed probesets (designed to antisense or intronic regions).

### Taqman and Transcriptomics Results

Good quality total RNA was isolated from 93 of the 96 SM explants, with sufficient RNA yield for transcriptomic analysis. Quantitative RT-PCR and open platform whole genome Affymetrix profiling was then performed. A single H1L2 treated biopsy failed to generate usable data by both platforms and was excluded from the data analysis.

Quantitative RT-PCR demonstrated significantly lower expression of IL-17A in the H1L2 treated explants compared to the Synagis™ IgG control. Although a signal of potential down regulation was detected in TNF-alpha expression when comparing treatment to control, this was not statistically significant.

| | | | | | H1L2 | | | Humira | | | Abatacept |
|---|---|---|---|---|---|---|---|---|---|---|---|
| | | | H1L2 | | Dunnett's | Humira | | Dunnett's | Abatacept | | Dunnett's |
| | | Uni Gene | Fold | H1L2 P | Adjusted | Fold | Humira | Adjusted | Fold | Abatacept | Adjusted |
| Gene | Entrez ID | ID | Change | Value | P Value | Change | P Value | P Value | Change | P Value | P Value |
| IL17A | 3605 | Hs.41724 | −1.79 | 0.037 | 0.096 | −1.40 | 0.209 | 0.457 | −1.27 | 0.440 | 0.7872 |
| TNF alpha | 7124 | Hs.241570 | −1.31 | 0.338 | 0.664 | −1.25 | 0.404 | 0.749 | −1.41 | 0.285 | 0.5866 |

Table 1 showing results from qPCR. Fold change for H1L2, Humira™, and abatacept compared to baseline (Synagis™ control IgG treatment group) with raw and Dunnett's adjusted p-values.

Hybridisation to Affymetrix U133plus2.0 Genechips, encompassing transcripts for 38,500 well characterised genes, identified significant changes (reliably detected probesets, P≦0.05) in the H1L2 treated explants compared to the Synagis™ IgG control for 3083 of the Affymetrix probesets. 4052 significant changes were identified in the Humira™ treated explants and 940 in the Abatacept treated explants. There is overlap between treatments, with approximately 30% of the probesets (1095) showing a significant change with H1L2 and showing a change with Humira™ treatment. There are, however, 1801 significant changes that are specific to H1L2 (P≦0.05) that do not show a significant change with Humira™ (P≧0.05). These microarray results are summarised in the Venn diagram of FIG. 2.

In order to identify genes that show a robust expression change with H1L2 but no change with Humira™, the data was filtered for changes≧|1.25| fold with H1L2 (P≦0.05) but no significant change (P>0.2) with Humira™. This generated a list of 233 probe sets (149 up-regulated, 84 down-regulated). For the purposes of reporting, all unannotated probe sets (no known target gene) have been removed, and only down regulated probe sets have been shown in table 2. Two up regulated probe sets, ITGA6 and Synapsin II, are, however, included in table 2.

Although probe set 243879_at has been mapped to Synapsin II, it seems more probable that the probe set in fact corresponds to TIMP4, for the following reasons. Firstly, Synapsin II shares a chromosomal location with TIMP4. Secondly, there have been no known links between Synapsin II and RA reported in the literature; however there have been reports of a protective role of TIMP4 in rodent RA models. Thirdly, internal mapping algorithms have identified 243879_at as representing TIMP4 instead of Synapsin II.

The gene for TIMP4 sits within an intronic region of the gene SYN2 but in the opposite direction. The Affymetrix probes sit in a region between the 3′ end of the coding region of TIMP4 and SYN2 exon, and the primers all read in the direction of TIMP4. Most of the ESTs that were used to make the consensus sequence contain polyTs which fits with them being reverse polyA tails at the end of the TIMP4 UTR region. In addition, the Ensembl Affymetrix mappings also map this probe set to TIMP4.

The incorrect mapping to Synapsin II has possibly occurred with NetAffx because NetAffx annotations are based on searching UniGene with EST IDs, and UniGene has pooled the TIMP4 ESTs together with SYN2 ESTs. Further, incorrect mapping is supported by the fact that these samples originated from RA synovial tissue, and Synapsin II shows enriched expression in the brain.

| | | | | H1L2 | | Humira | |
|---|---|---|---|---|---|---|---|
| | | | Entrez | Fold | H1L2 P | Fold | Humira P |
| Probe Set ID | Gene Symbol | UniGene ID | Gene | Change | Value | Change | Value |
| 213955_at | MYOZ3 | Hs.91626 | 91977 | −2.37 | 0.0009 | −1.06 | 0.802712 |
| 231798_at | NOG | Hs.248201 | 9241 | −1.64 | 0.02202 | −1.20 | 0.390346 |
| 201169_s_at | BHLHB2 | Hs.171825 | 8553 | −1.57 | 0.01243 | −1.19 | 0.319864 |
| 214219_x_at | MAP4K1 | Hs.95424 | 11184 | −1.54 | 0.0012 | −1.13 | 0.336725 |
| 230319_at | C4orf31 | Hs.90250 | 79625 | −1.53 | 0.00459 | −1.10 | 0.491654 |
| 206254_at | EGF | Hs.419815 | 1950 | −1.51 | 0.00509 | −1.14 | 0.332697 |
| 206239_s_at | SPINK1 | Hs.407856 | 6690 | −1.50 | 0.00852 | −1.02 | 0.893265 |
| 229909_at | B4GALNT3 | Hs.504416 | 283358 | −1.48 | 0.01316 | −1.17 | 0.310437 |
| 219747_at | C4orf31 | Hs.90250 | 79625 | −1.48 | 0.0074 | −1.09 | 0.540713 |
| 222333_at | ALS2CL | Hs.517937 | 259173 | −1.46 | 0.00444 | −1.14 | 0.303389 |
| 207113_s_at | TNF | Hs.241570 | 7124 | −1.45 | 0.01942 | 1.02 | 0.882459 |
| 229854_at | OBSCN | Hs.231655 | 84033 | −1.43 | 0.02008 | −1.09 | 0.560875 |
| 229596_at | AMDHD1 | Hs.424907 | 144193 | −1.42 | 0.02756 | −1.10 | 0.542461 |
| 238309_x_at | LOC441119 | Hs.348792 | 441119 | −1.40 | 0.02139 | −1.05 | 0.736212 |
| 244261_at | IL28RA | Hs.221375 | 163702 | −1.39 | 0.01628 | −1.04 | 0.780458 |
| 241871_at | CAMK4 | Hs.591269 | 814 | −1.39 | 0.00593 | 1.04 | 0.736145 |
| 232602_at | WFDC3 | Hs.419126 | 140686 | −1.38 | 0.02297 | −1.04 | 0.757134 |
| 236717_at | LOC165186 | Hs.525977 | 165186 | −1.38 | 0.04321 | −1.20 | 0.224875 |
| 1564121_at | ABR | Hs.159306 | 29 | −1.37 | 0.00685 | −1.15 | 0.204435 |
| 207987_s_at | GNRH1 | Hs.82963 | 2796 | −1.36 | 0.01788 | −1.12 | 0.365604 |
| 236863_at | C17orf67 | Hs.158851 | 339210 | −1.35 | 0.01288 | −1.15 | 0.228871 |
| 206634_at | SIX3 | Hs.567336 | 6496 | −1.35 | 0.0447 | −1.06 | 0.698929 |
| 213213_at | DIDO1 | Hs.517172 | 11083 | −1.34 | 0.00437 | −1.09 | 0.38518 |
| 1566809_a_at | PCBP3 | Hs.474049 | 54039 | −1.34 | 0.03182 | −1.16 | 0.251868 |
| 1569723_a_at | SPIRE2 | Hs.461786 | 84501 | −1.34 | 0.02375 | 1.06 | 0.620334 |
| 230702_at | C8orf16 | Hs.646175 | 83735 | −1.33 | 0.0073 | −1.09 | 0.388174 |
| 40020_at | CELSR3 | Hs.631926 | 1951 | −1.33 | 0.0257 | −1.16 | 0.21849 |
| 1552364_s_at | MSI2 | Hs.585782 | 124540 | −1.32 | 0.01209 | −1.14 | 0.221172 |
| 236176_at | LOC645757 | Hs.18768 | 645757 | −1.32 | 0.03249 | 1.02 | 0.861687 |
| 207291_at | PRRG4 | Hs.471695 | 79056 | −1.32 | 0.04528 | −1.17 | 0.227066 |
| 209480_at | HLA-DQB1 | Hs.409934 | 3119 | −1.31 | 0.03759 | 1.01 | 0.921824 |
| 235514_at | SASP | Hs.556025 | 151516 | −1.31 | 0.03622 | −1.12 | 0.356644 |
| 203673_at | TG | Hs.584811 | 7038 | −1.31 | 0.01904 | −1.14 | 0.21712 |
| 1556809_a_at | LOC121906 | Hs.646603 | 121906 | −1.30 | 0.01842 | 1.00 | 0.964444 |
| 215233_at | PTDSR | Hs.514505 | 23210 | −1.30 | 0.01944 | −1.06 | 0.593645 |
| 244429_at | LOC643264 | Hs.434392 | 643264 | −1.30 | 0.01358 | −1.08 | 0.445447 |
| 217149_x_at | TNK1 | Hs.203420 | 8711 | −1.30 | 0.00471 | −1.09 | 0.318631 |
| 221887_s_at | DFNB31 | Hs.93836 | 25861 | −1.30 | 0.02054 | −1.14 | 0.231875 |
| 204857_at | MAD1L1 | Hs.209128 | 8379 | −1.30 | 0.03625 | 1.02 | 0.837342 |
| 226415_at | KIAA1576 | Hs.461405 | 57687 | −1.30 | 0.04249 | −1.12 | 0.357255 |
| 215349_at | LOC643376 | Hs.645350 | 643376 | −1.30 | 0.01244 | −1.08 | 0.42367 |
| 241396_at | C10orf6 | Hs.447458 | 55719 | −1.29 | 0.02034 | −1.13 | 0.252889 |
| 207072_at | IL18RAP | Hs.158315 | 8807 | −1.29 | 0.03062 | −1.15 | 0.214398 |
| 207075_at | NLRP3 | Hs.159483 | 114548 | −1.29 | 0.02496 | −1.15 | 0.211502 |
| 206723_s_at | EDG4 | Hs.122575 | 9170 | −1.28 | 0.03007 | −1.02 | 0.851184 |
| 1560512_at | INADL | Hs.478125 | 10207 | −1.28 | 0.0217 | −1.13 | 0.23286 |
| 230981_at | CATSPER3 | Hs.631804 | 347732 | −1.28 | 0.00923 | −1.03 | 0.765769 |
| 243264_s_at | SGK3 /// | Hs.545401 | 23678 /// | −1.28 | 0.02208 | −1.07 | 0.524689 |
| | C8orf44 | | 56260 | | | | |
| 244691_at | SETD5 | Hs.288164 | 55209 | −1.27 | 0.00572 | −1.09 | 0.302526 |
| 223912_s_at | CLN8 | Hs.127675 | 2055 | −1.27 | 0.0407 | −1.15 | 0.227129 |
| 204902_s_at | ATG4B /// | Hs.283610 | 23192 /// | −1.27 | 0.00495 | −1.08 | 0.333602 |
| | LOC727737 | | 727737 | | | | |
| 203332_s_at | INPP5D | Hs.601911 | 3635 | −1.27 | 0.02614 | −1.12 | 0.267191 |
| 235533_at | COX19 | Hs.121593 | 90639 | −1.27 | 0.04113 | −1.03 | 0.785879 |
| 224491_at | APOL4 | Hs.115099 | 80832 | −1.26 | 0.00686 | 1.00 | 0.95589 |
| 219095_at | PLA2G4B | Hs.198161 | 8681 | −1.26 | 0.01329 | −1.09 | 0.333591 |
| 226622_at | MUC20 | Hs.599259 | 200958 | −1.26 | 0.03156 | −1.09 | 0.417525 |
| 212679_at | TBL2 | Hs.647044 | 26608 | −1.26 | 0.04517 | −1.12 | 0.302412 |
| 209603_at | GATA3 | Hs.524134 | 2625 | −1.25 | 0.03978 | −1.14 | 0.212237 |
| 242790_at | SNF8 | Hs.127249 | 11267 | −1.25 | 0.03265 | 1.02 | 0.827876 |
| 47553_at | DFNB31 | Hs.93836 | 25861 | −1.25 | 0.01782 | −1.10 | 0.313534 |
| 1561334_at | LOC285181 | Hs.434525 | 285181 | −1.25 | 0.04721 | −1.12 | 0.315361 |
| 228320_x_at | CCDC64 | Hs.369763 | 92558 | −1.25 | 0.02578 | −1.11 | 0.293632 |
| 215177_s_at | ITGA6 | Hs.133397 | 3655 | 1.25 | 0.02393 | 1.04 | 0.69209 |
| 243879_at | SYN2 | Hs.445503 | 6854 | 1.39 | 0.02728 | 1.00 | 0.993388 |

Table 2 showing H1L2 and Humira™ fold changes and p values from microarray study. All probe sets were mapped to genes using Affymetrix standard annotation through NetAffx (www.affymetrix.com). The table has been filtered to show only probe sets with an expression change ≦−1.25 with H1L2, but not Humira (P≦0.05 H1L2 vs. Synagis, P>0.2 Humira™ vs. Synagis). Data for 2 up-regulated genes; ITGA6 and Synapsin II (TIMP4—see above) has also been included.

A significant change was observed in TNF-α expression by microarray but not TaqMan when comparing H1L2 treatment to control. As both technologies were performed on the same samples, this can be explained by more technical variability and a smaller detected fold change by TaqMan (TaqMan normalisation uses less data points to normalise than microarray). Power calculations were performed on both data to provide more details. The measured difference between treatments in TNF-α expression by TaqMan was 1.31, and the power calculation indicated there was a 20% chance that we would reach significance (P<0.05) for a 1.35 fold change. Therefore it is very unlikely that a 1.31 fold change would be detected as significant change by TaqMan. Conversely, the measured difference between treatments in TNF-α expression by microarray was 1.45, and the power calculation indicated there was a 70% chance that we would reach significance (P<0.05) for a 1.45 fold change. Therefore it is more likely that a 1.45 fold change would be detected as significant change by microarray.

### Conclusions

It is apparent from our experiments and current knowledge that H1L2 modulates the expression of a different subset of genes compared with the TNF-α antagonist adalimumab (Humira™) and the T-cell co-stimulation antagonist abatacept (see FIG. 2). 1801 significant changes in gene expression, specific to H1L2 treatment (P≦0.05) and hence not observed with either adalimumab or abatacept (P>0.05), were detected: this number of changes corresponds to 58% of the total number of changes that were detected with H1L2 treatment.

It is clear from the above data that TNF-α antagonists, T-cell co-stimulation antagonists and IL-18 antagonists affect the expression of different gene subsets. A core set of genes is affected by all three types of antagonists; there are genes that are affected by two out of the three antagonists, but not the third; and there are genes which are affected by only one of the antagonists. Where there are genes linked to a disease state and these genes are specifically targeted by one of the antagonists, but not the others, the potential for treating subjects that are non-responsive or refractory to the other two antagonists arises. These results indicate, for the first time, that treatment with IL-18 antagonists is likely to be beneficial in those patients that are non-responsive or refractory to treatment with TNF-α antagonists and T-cell co-stimulation antagonists.

Surprisingly, IL-17A, a cytokine implicated in the inflammatory response and associated with a number of autoimmune diseases including RA, is among the subset of genes specifically modulated by the anti-IL-18 antibody H1L2, but not by adalimumab or abatacept (see table 1).

IL-17A is produced mainly by CD4+ T cells. The main effects of IL-17A are proinflammatory, including production by macrophages of proinflammatory mediators (TNF-α, IL-1β, IL-6, IL-12 and PGE2) (Jovanovic et al., 1998) and production by fibroblasts and keratinocytes of IL-16 and IL-8. Injecting recombinant IL-17A into joints has been reported to cause joint inflammation and cartilage destruction (Dudler et al., 2000). In the mouse cartilage induced arthritis (CIA) model, injection of an adenovirus encoding IL-17A increases disease severity and induces production of the receptor activator of nuclear factor κB ligand (RANKL) through mechanisms independent from IL-1 (Lubbers et al., 2000). IL-17A is found in the rheumatoid synovium, particularly those areas containing large numbers of T-cells. These cells appear to contribute directly to the destructive process leading to bone loss and cartilage degradation (Chabaud et al., 1999).

Blockade of IL-17A in vivo suppresses inflammation, joint destruction and disease progression in a number of arthritic models. Use of IL-17A receptor (IL-17R) Fc fusion proteins have demonstrated suppression of joint damage at the macroscopic level in murine CIA (Nakae et al., 2003) and also by histological analysis in rat adjuvant induced arthritis (AIA) models of arthritis. Use of commercial neutralising antibodies to IL-17A (rat anti-mouse) and IL-17R (rat anti-mouse) have demonstrated inhibition of swelling and arthritis onset in an infectious model of arthritis (Nakae et al., 2003). Rabbit anti-mouse polyclonal antibodies have been used in murine models of arthritis demonstrating decreased severity of joint damage and cartilage destruction and reduction of levels of pro-inflammatory mediators including RANKL, IL-1β and IL-6 (Lubberts et al., 2004). More recently Hoeve et al., (2006) described divergent effects of IL-12 and IL-23 on the production of IL-17A by human T-cells.

Of particular relevance to the treatment of RA, expression of the following genes is specifically downregulated with H1L2 treatment but not with Humira™ treatment: noggin, EGF, TNF-α and IL-18RAP (see table 2).

Noggin has been involved in cartilage damage in a mouse CIA model (Lories et al., 2006). EGF binds selectively to ADAMTS7, a metalloproteinase that directly binds to and degrades cartilage oligomeric matrix protein (Liu et al., 2006). EGF promotes chondrocyte proliferation during skeletal development and accumulates in the synovial fluid in RA (Bonassar et al., 1997). EGF increases DNA synthesis and the production of MMP-1 and MMP-3 (Domeij et al., 2002). TNF-α is a proinflammatory cytokine produced by many cell types (blood monocytes, macrophages, mast cells and endothelial cells) that plays a key role in the pathogenesis of multiple autoimmune and nonautoimmune disorders (Atzeni et al., 2007). IL-18RAP (IL-18Rβ) is essential for IL-18 signal transduction and ligand binding affinity to IL-18Rα receptor chain (Fizsher et al., 2007).

Table 2 also shows that integrin-α6 and TIMP-4 (243879_at mapped as Synapsin II), both RA associated genes, are specifically upregulated with H1L2 treatment but not with Humira™ treatment.

Integrin-α6 is at very low levels in hyperplastic synovial membrane whereas this integrin is well expressed in noninflammatory synovium. This integrin is enhanced by TGF-β and downregulated by a combination of TNF-α and interferon-γ (Pirila et al., 1996). TIMP-4 is a tissue inhibitor of matrix metalloproteinases. It has been reported that treatment with TIMP-4 improves disease scores in a model of rat arthritis (Ramamurthy et al., 2005).

These conclusions are based on our current findings. Knowledge on the exact mechanisms involved, however, may be incomplete.

### References

Atzeni et al. Potential target of infliximab in autoimmune and inflammatory diseases. Autoimmunity Reviews 6(8): 529-36 (2007).
Barrera et al. Effects of treatment with a fully human anti-tumour necrosis factor alpha monoclonal antibody on the local and systemic homeostasis of interleukin-1 and TNF alpha in patients with rheumatoid arthritis. Ann Rev. Med. 60: 660-669 (2001).
Bonassar, L. J. et al. Interaction of Epidermal Growth Factor and Insulin-like Growth Factor-I in the Regulation of Growth Plate Chondrocytes. Experimental Cell Research 234(1): 1-6 (1997).
Bush K. A. et al. Reduction of joint inflammation and bone erosion in rat adjuvant arthritis by treatment with interleukin-17 receptor IgG1 Fc fusion protein. Arthritis & Rheumatism 46(3): 802-5 (2002).
Carlin et al. A 50% reduction in the Psoriasis Area and Severity Index (PASI 50) is a clinically significant endpoint in the assessment of psoriasis. J. Am. Acad. Dermatol. 50(6): 859-866 (2004).
Chabaud et al. Human interleukin 17: a T cell derived proinflammatory cytokine produced by the rheumatoid synovium. Arthritis Rheum. 42: 963-70 (1999).
Choy et al. Efficacy of a novel PEGylated humanised anti-TNF fragment (CDP870) in patients with rheumatoid arthritis: a phase II double-blinded, randomized, dose-escalating trial. Rheumatology 41: 1133-1137 (2002).
Choy E. H, Panayi G. S. Cytokine pathways and joint inflammation in rheumatoid arthritis. N. Engl. J. Med. 344 (12): 907-916 (2001).
Davis et al. Treatment of rheumatoid arthritis with PEGylated recombinant soluble tumour necrosis factor receptor type I: a clinical update. Ann Rheum. Dis. 59 (Suppl. 1): 41-43 (2000).
Domeij et al. Signal pathways involved in the production of MMP-1 and MMP-3 in human gingival fibroblasts. European Journal of Oral Sciences 110(4): 302-6 (2002).
Dudler et al. Effect of interleukin 17 on proteoglycan degradation in murine knee joints. Ann Rheum. Dis. 59: 529-32 (2000).
Durez P., Van Der Bosch F., Corluy L. et al. A dose adjustment in patients with rheumatoid arthritis not optimally responding to a standard dose of infliximab of 3 mg/kg every 8 weeks can be effective: a Belgian prospective study. Rheumatology (Oxford) 44(4): 465-468 (2005).
Feldman et al. Psoriasis assessment tools in clinical trials. Ann Rheum. Dis. 64: (suppl. II): ii65-ii68 (2005).
Fizsher et al. Identification of IL-18RAP mRNA truncated splice variants in human testis and the other human tissues. Cytokine 39(3): 178-63 (2007).
Fransen, J. The Disease Activity Score and the EULAR response criteria. Clinical and Experimental Rheumatology. 23 (Suppl. 39): S93-S99 (2005).
Hoeve et al. Divergent effects of IL-12 and IL-23 on the production of IL-17 by human T cells. Eur. J. Immuno. 36: 661-670 (2006).
Jovanovic et al. IL17 stimulates the production and expression of proinflammatory cytokines, IL-1 beta and TNF-alpha, by human macrophages. J. Immunol. 160: 3513-21 (1998).
Kabat et al. Sequences of Proteins of Immunological Interest, 4th Ed., U.S. Department of Health and Human Services, National Institutes of Health (1987).
Keane et al. Tuberculosis Associated with Infliximab, a Tumor Necrosis Factor α-Neutralizing Agent. N. Engl. J. Med. 345: 1098-1104 (2001).
Kremer, J. M. Rational use of new and existing disease-modifying agents in rheumatoid arthritis. Ann Interm. Med. 134 (8): 695-706 (2001).
Liu, Chaun-Ju. Kong et al. ADAMTS-7: a metalloproteinase that directly binds to and degrades cartilage oligomeric matrix protein. F.A.S.E.B. Journal 20(7): 988-90 (2006).
Lories et al. Noggin haploinsufficiency differentially affects tissue responses in destructive and remodeling arthritis. Arthritis & Rheumatism 54(6): 1736-46 (2006).
Lubbers et al. Local overexpression of IL-17 in the knee joint of collagen type II immunised mice promotes collagen arthritis and aggravates joint destruction independent of IL-1. Arthritis Rheum. 43 (Suppl. 9): 170 (2000).
Lubberts E. et al. Treatment with a neutralizing anti-murine interleukin-17 antibody after the onset of collagen-induced arthritis reduces joint inflammation, cartilage destruction, and bone erosion. Arthritis Rheum. 50(2): 650-9 (2004).
Lundquist, L. M. Abatacept: a novel treatment for rheumatoid arthritis. Expert Opin. Pharmacother. 8(14): 2371-2379 (2007).
Maini and Taylor. Anti-cytokine therapy in rheumatoid arthritis. Annu Rev. Med. 51: 207-229 (2000).
Moreland et al. Abatacept. Nature Reviews Drug Discovery 5: 185-186 (2006).
Nakae et al. IL-17 production from activated T cells is required for the spontaneous development of destructive arthritis in mice deficient in IL-1 receptor antagonist. Proc. Natl. Acad. U.S.A. 100, 5986-90 (2003).
Pirila and Heino. Altered integrin expression in rheumatoid synovial lining type B cells: in vitro cytokine regulation of alpha 1 beta 1, alpha 6 beta 1, and alpha v beta 5 integrins. J. Reumatol. 23(10): 1691-8 (1996).
Pollard et al. Rheumatoid arthritis: non-tumor necrosis factor targets. Curr. Opin. Rheumatol. 17: 242-246 (2005).
Ramamurthy et al. Experimental Arthritis in Rats Induces Biomarkers of Periodontitis Which Are Ameliorated by Gene Therapy With Tissue Inhibitor of Matrix Metalloproteinases. J. Periodontology 76(2): 229-33 (2005).
Rothe et al. Active stage of autoimmune diabetes is associated with the expression of a novel cytokine, IGIF, which is located near Idd2. J. Clin. Invest. 99: 469 (1997).
Smolen et al. Therapeutics Strategies for Rheumatoid Arthritis. Nature Reviews Drug Discovery 2: 473-488 (2003).
Sostegni et al. Review article: Crohn's disease: monitoring disease activity. Aliment Pharmacol. Ther. 17 (Suppl. 2): 11-17 (2003).
Taniguchi et al. Characterization of anti-human interleukin-18 (IL-18)/interferon-γ-inducing factor (IGIF) monoclonal antibodies and their application in the measurement of human IL-18 by ELISA. Immunol. Methods 206: 107 (1997).
Weisman et al. Abatacept (CTLA4Ig; BMS-188667) inhibits T-cell activation and the subsequent activation of inflammatory cell types, as demonstrated by sustained reductions in multiple inflammatory biomarkers. Ann Rheum. Dis. 63: Suppl. 1:i138 abstract (2004).
Wildbaum et al. Neutralizing Antibodies to IFN-γ-Inducing Factor Prevent Experimental Autoimmune Encephalomyelitis. J. Immunol. 161: 6368 (1998).

SEQUENCE LISTING
(CDRH1)
SEQ ID NO: 1
GYYFH
(CDRH2)
SEQ ID NO: 2
RIDPEDDSTKYAERFKD
(CDRH3)
SEQ ID NO: 3
WRIYRDSSGRPFYVMDA
(CDRL1)
SEQ ID NO: 4
LASEDIYTYLT
(CDRL2)
SEQ ID NO: 5
GANKLQD
(CDRL3)
SEQ ID NO: 6
LQGSKFPLT
(H1)
SEQ ID NO: 7
QVQLVQSGAEVKKPGASVKVSCKVSGEISTGYYFHWVRQAPGKGLEWMG
RIDPEDDSTKYAERFKDRVTMTEDTSTDTAYMELSSLRSEDTAVYYCTT
WRIYRDSSGRPFYVMDAWGQGTLVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK
(H2)
SEQ ID NO: 8
QVQLVQSGAEVKKPGASVKVSCKVSGEISTGYYFHWVRRRPGKGLEWMG
RIDPEDDSTKYAERFKDRVTMTEDTSTDTAYMELSSLRSEDTAVYYCTT
WRIYRDSSGRPFYVMDAWGQGTLVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK
(H3)
SEQ ID NO: 9
QVQLVQSGAEVKKPGASVKVSCKVSGEISTGYYFHFVRRRPGKGLEWMG
RIDPEDDSTKYAERFKDRVTMTADTSTDTAYMELSSLRSEDTATYFCTT
WRIYRDSSGRPFYVMDAWGQGTLVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK
(L1)
SEQ ID NO: 10
DIQMTQSPSSVSASVGDRVTITCLASEDIYTYLTWYQQKPGKAPKLLIY
GANKLQDGVPSRFSGSGSGTDFTLTISSLQPEDFATYYCLQGSKFPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(L2)
SEQ ID NO: 11
DIQMTQSPSSVSASVGDRVTITCLASEDIYTYLTWYQQKPGKAPKLLIY
GANKLQDGVPSRFSGSGSGTDYTLTISSLQPEDFATYYCLQGSKFPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(L3)
SEQ ID NO: 12
DIQMTQSPSSVSASVGDRVTITCLASEDIYTYLTWYQQKPGKAPQLLIY
GANKLQDGVPSRFSGSGSGTDYTLTISSLQPEDEGDYYCLQGSKFPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(IL-18)
SEQ ID NO: 13
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMFTVQNED
(TaqMan forward primer for GADPH
(Hs. 544577, 2597))
SEQ ID NO: 14
CAAGGTCATCCATGACAACTTTG
(TaqMan reverse primer for GADPH
(Hs. 544577, 2597))
SEQ ID NO: 15
GGGCCATCCACAGTCTTCTG
(TaqMan probe sequence for GADPH
(Hs. 544577, 2597))
SEQ ID NO: 16
ACCACAGTCCATGCCATCACTGCCAT
(TaqMan forward primer for Bactin
(Hs. 520640, 60))
SEQ ID NO: 17
GAGCTACGAGCTGCCTGACTG
(TaqMan reverse primer for Bactin
(Hs. 520640, 60))
SEQ ID NO: 18
GTAGTTTCGTGGATGCCACAGGACT
(TaqMan probe sequence for Bactin
(Hs. 520640, 60))
SEQ ID NO: 19
CATCACCATTGGCAATGAGCGGTTTCC
(TaqMan forward primer for Cyclophilin
(Hs. 356331, 5478))
SEQ ID NO: 20
CATCTGCACTGCCAAGACTGA
(TaqMan reverse primer for Cyclophilin
(Hs. 356331, 5478))
SEQ ID NO: 21
CCACAATATTCATGCCTTCTTTCA
(TaqMan probe sequence for Cyclophilin
(Hs. 356331, 5478))
SEQ ID NO: 22
CCAAACACCACATGCTTGCCATCCA
(TaqMan forward primer for TNF-α
(Hs. 241570, 7124))
SEQ ID NO: 23
GGTGCTTGTTCCTCAGCCTC
(TaqMan reverse primer for TNF-α
(Hs. 241570, 7124))
SEQ ID NO: 24
CAGGCAGAAGAGCGTGGTG
(TaqMan probe sequence for TNF-α
(Hs. 241570, 7124))
SEQ ID NO: 25
CTCCTTCCTGATCGTGGCAGGCG
(SybrMan forward primer sequence for IL17A
(Hs. 41724, 3605))
SEQ ID NO: 26
CGCAATGAGGACCCTGAGA
(SybrMan reverse primer sequence for IL17A
(Hs. 41724, 3605))
SEQ ID NO: 27
ACGTTCCCATCAGCGTTGA

1. A method of treating an autoimmune disease in a subject that is non-responsive or refractory to treatment with a TNF-α antagonist and/or a T-cell co-stimulation antagonist, said method comprising the step of administering to the subject a therapeutically effective amount of an IL-18 antagonist.

2. The method of claim 1, wherein the autoimmune disease is selected from the group of: inflammatory bowel disease (IBD), psoriasis, type I diabetes, MS, and an arthritic disease.

3. The method of claim 1, wherein the autoimmune disease is rheumatoid arthritis (RA).

4. The method of claim 1, wherein the TNF-α antagonist is selected from the group of: infliximab (Remicade™), etanercept (Enbrel™), adalimumab (Humira™), CDP571, CDP870, and CNTO148 (golimumab).

5. The method of claim 1, wherein the TNF-α antagonist is adalimumab (Humira™).

6. The method of claim 1, wherein the T-cell co-stimulation antagonist is abatacept (Orencia™).

7. The method of claim 1, wherein the IL-18 antagonist is an antibody immunospecific for IL-18.

8. The method of claim 7, wherein the antibody is a humanised anti-IL-18 antibody comprising a heavy chain and light chain having the following complementarity determining regions (CDRs):

| | |
|---|---|
| CDRH1: | SEQ ID NO: 1 |
| CDRH2: | SEQ ID NO: 2 |
| CDRH3: | SEQ ID NO: 3 |
| CDRL1: | SEQ ID NO: 4 |
| CDRL2: | SEQ ID NO: 5 |
| CDRL3: | SEQ ID NO: 6. |

9. The method of claim 8, wherein one or more of the CDRs is replaced by a variant thereof, each variant CDR containing 1 or 2 amino acid substitutions, insertions or deletions.

10. The method of claim 8, wherein the antibody is a humanised anti-IL-18 antibody comprising a heavy chain of SEQ ID NO:7 (H1) and a light chain of SEQ ID NO: 11 (L2).

11. The method of as claimed in claim 7, wherein the antibody is an antibody that competes with an antibody comprising a heavy chain having the sequence set forth in SEQ ID NO:7 (H1) and a light chain having the sequence set forth in SEQ ID NO:11 (L2) for binding to human IL-18 in an ELISA assay.

12. The method of as claimed in claim 7, wherein the antibody comprises heavy and light chains comprising polypeptides which are at least 90%, 95%, 96%, 97%, 98% or 99% identical to the amino acid sequences of SEQ ID NO:7 (H1) and SEQ ID NO:11 (L2), respectively, wherein said antibody binds human IL-18.

13. The method of claim 1, wherein IL-17A expression is down-regulated.

14. An IL-18 antagonist for use in the treatment of an autoimmune disease in a subject that is non-responsive or refractory to treatment with a TNF-α antagonist and/or a T-cell co-stimulation antagonist.

15. An IL-18 antagonist for use in down-regulating IL-17A expression or activity in a subject afflicted with an autoimmune disease.
